(19) United States
(12) Patent Application Publication
(10) Pub. No.: US 2007/0287911 A1
(43) Pub. Date: Dec. 13, 2007

(54) Method and device for navigating and positioning an object relative to a patient

(75) Inventors: Markus Haid, Stuttgart (DE); Urs Schneider, Stuttgart (DE); Kai von Luebtow, Stuttgart (DE)

Correspondence Address: BRINKS HOFER GILSON & LIONE, P.O. BOX 10395, CHICAGO, IL, 60610, US

(73) Assignee: Fraunhofer-Gesellschaft zur Forderung der angewandten, Munchen (DE)

(21) Appl. No.: 11/809,682

(22) Filed: Jun. 1, 2007

Related U.S. Application Data
(63) Continuation of application No. PCT/EP2005/012473, filed on Nov. 22, 2005.

(30) Foreign Application Priority Data
Dec. 1, 2004 (DE) 10 2004 057 933.4

(51) Int. Cl. A61B 5/05; A61B 19/00

(52) U.S. Cl. 600/429; 606/130

(57) ABSTRACT

The disclosure relates to a method and a device for navigating and positioning an object relative to a patient during surgery in an operating room. According to the disclosure, the position and orientation of the object and the patient in the room or a respective area of the patient relative to a reference system are determined quasi continuously in accordance with a scanning rate by means of a three-dimensional inertial sensor system, the momentary position and orientation of the object relative to the patient are determined therefrom, said position and orientation are compared to a desired predetermined position and orientation, and an indication is made as to how the position of the object has to be modified in order to reach the desired predetermined position and orientation.

### CROSS-REFERENCE TO RELATED APPLICATIONS

This application is a continuation of International Application No. PCT/EP2005/012473 filed on Nov. 22, 2005, which claims the benefit of German Patent Application No. 10 2004 057 933.4 filed Dec. 1, 2004. The disclosures of the above applications are incorporated herein by reference.

### FIELD

The present disclosure relates to a method and a device for navigating and positioning an object relative to a patient during surgery in an operating room.

### BACKGROUND

The statements in this section merely provide background information related to the present disclosure and may not constitute prior art.

By way of example, during the navigating and positioning process the hip prosthesis may be arranged in a precisely predetermined position relative to the femur of the patient. Up to now, correct positioning has been checked optically by means of cameras in the operating room. Marks that can be recognized optically have been applied to the object or device provided for navigation. These marks have also been applied to the patient. Although these systems operate with the required precision, they also entail many disadvantages. The system required for this purpose, with several cameras and a control panel, is very expensive, its cost amounting to tens of thousands of euros. Such a system operates based on references, i.e. on principle, it is only for stationary use. If the system is used elsewhere, the cameras necessary for this process have to be mounted again in exactly predetermined places. It is further disadvantageous that a limited workspace measuring only approximately 1 m is available. An especially problematic disadvantage is shading. The system is only operative if the marks applied for this purpose can be captured simultaneously by all the required cameras. If surgical staff are standing between such a mark and a camera, or if other surgical devices are in that position, the system will not operate.

### SUMMARY

The task of the present disclosure is to improve a method and device of the type described above such that the aforesaid disadvantages do not occur or are overcome to a large extent.

This task is achieved by a method and a device according to the characteristics described in the independent claims 1 and 2.

The application of the method according to the disclosure and the device according to the disclosure allows the object, e.g. the part of a prosthesis or surgical device, to be placed in a predetermined position on the patient, in other words, the surgical staff can modify the position and orientation of the involved object so that it reaches the predetermined position relative to the patient or relative to an area of the patient. The claimed device is far less expensive than the method of optical capture described at the beginning. The system according to the present disclosure is essentially portable; except for the sensor devices that can be applied to the object and the patient, there is no need for large equipment, which would require complex stationary mounting in precisely predetermined positions. The sensor devices, which contain three-dimensional inertial sensor technology, can also be miniaturized to a large extent, and may be made the size of a few millimeters. These sensor devices can be attached to and again detached from the aforesaid object in a predetermined position and orientation, on the one hand, and in a likewise predetermined position on the patient, on the other hand. For this purpose, the sensor devices advantageously have an orientation aid that can preferably be recognized visually, which allows correct fixation on the object and patient, respectively.

It is also advantageous for the sensor devices to have fastening elements for the detachable fixation of the sensor device on the object and the patient. These can be fastening elements executed as clamps or clips, for instance.

To compare position data from values measured by the first sensor device and values measured by the second sensor device in the course of the procedure, it is necessary only to reference the sensor devices at the beginning of positioning by bringing both sensor devices to a standstill in a common place or in two places oriented to each other in a known predetermined orientation. On this basis, the displacement of the sensor devices is determined using the three-dimensional sensors, and is entered for further data processing.

The first and particularly also the second sensor device preferably have three acceleration sensors, whose signals may be used to calculate translational movements, and also three rotational speed sensors, whose measured values may be used for orientation in the room.

In addition to the rotational speed sensors, magnetic field sensors for calculating the orientation of the object in the room may advantageously be provided. The magnetic field sensors acquire terrestrial magnetic field components and can provide information on the orientation of the sensor device in the room.

In such cases it is advantageous if means are provided for comparing the orientation in the room determined on the basis of the measured values acquired by the magnetic field sensors with the orientation in the room determined on the basis of the measured values acquired by the rotational speed sensors. In addition, an acceleration sensor for measuring gravitational acceleration may be provided, which can likewise be used to calculate the orientation of the considered sensor device in the room.

It is further advantageous if the means of calculation of the device according to the present disclosure have means for executing a quaternion algorithm known as such from DE 103 12 154 A1.

It is also advantageous if the means of calculation have means for applying a compensation matrix determined and stored prior to the start of positioning, said compensation matrix allowing for a deviation in the axial orientation of the three rotational speed sensors from an assumed orientation of the axes toward each other, and, when used, compensating for errors resulting from the calculation of the rotation angles.

It is additionally advantageous if the means of calculation disclose means for implementing a Kalman filter algorithm.

Because inertial sensors provide measured values referred to the acceleration processes, these measured values are integrated twice for the determination of position data in the case of acceleration sensors, and are integrated once in the case of rotational speed sensors. During the course of these integration processes, errors prior to and after integration are added up by integration. It is insofar advantageous that the process and the applied apparatus are executed such that, when the apparatus is in a position of rest for a certain period of time, prior to and also after the data determination an offset value of the output signal of the rotational speed sensors is determined and afterwards subtracted until the next determination of this offset value for the respective rotational speed sensors, so that it is not included in the integration. This ensures that a new, current offset value is constantly determined in order to achieve maximal precision.

It has also been found that the inevitable constructive deviation in the axial orientation of the three rotational speed sensors from an assumed orientation very rapidly results in an imprecise calculation of the rotational speeds. By compensating for this error with the compensation matrix to be determined for the involved sensor device, increased precision to comply with the requirements can be achieved. To determine the compensation matrix, a sensor device used for this purpose is rotated around each axis prior to object tracing, while the other two axes are inoperative. Based on the signals of the rotational speed sensors acquired in this way, the compensation matrix is calculated and stored in a memory of the respective device. An industrial robot may be used for the rotational actuation of the sensor device. Thus, the 3×3 non-orthogonality matrix can be acquired consecutively via rotation around the individual space axes.

$$\underline{\underline{N}} = ( \begin{matrix}{N}_{11} & {N}_{12} & {N}_{13} \\ {N}_{21} & {N}_{22} & {N}_{23} \\ {N}_{31} & {N}_{32} & {N}_{33}\end{matrix} ) \qquad ( 1 )$$

In an ideal system, the secondary diagonal elements of the non-orthogonality matrix in the equation would be equal to 0. The imprecision results from manufacturing imprecision that leads to the axes of the rotational speed sensors being in neither a predetermined orientation relative to the sensor device casing nor arranged exactly orthogonal to each other.

If, as mentioned above, an offset value is determined when the apparatus is found to be stopped for a certain time, this denotes the determination of a drift vector, namely for three rotational speed sensors arranged preferably orthogonally to one another and for three acceleration sensors arranged preferably orthogonally to one another. Thus, a matrix D can be determined, whose rows are the offsets of the individual sensors. They are preferably determined when object tracing is enabled and afterward always when a rest period is detected, and are taken as a basis for further data processing.

$$\underline{\underline{D}} = ( \begin{matrix}{D}_{1} \\ {D}_{2} \\ {D}_{3}\end{matrix} ) \qquad ( 2 )$$

The precision of the determination of the orientation is also increased by an embodiment according to the present disclosure such that at each data acquisition and determination of the three rotation angles, a quaternion algorithm of the type described below is applied to the three rotation angles in order to calculate the orientation of the object in the room.

The improvement achieved in this way is based on the following circumstance: If the infinitesimal rotation angles around each axis, which can be obtained by simple integration at each infinitesimal sensing step, i.e. at acquisition of the measured data, for the purpose of determining the change in orientation of the object, were taken such that the rotations around the axes were consecutive, this would result in an error. This error occurs because the data measured by the three sensors are taken at the same time, because rotation normally occurs and is determined simultaneously around three axes. If, however, the changes in position of the three determined rotation angles were considered consecutively as rotations around the respective axes to determine the change in position, an error would result from the rotation around the second and third axes, because these axes would have already been placed incorrectly in another orientation in the course of the first rotation. This is counteracted by applying the quaternion algorithm to the three rotation angles. Thus, the three rotations are replaced by a single transformation. The quaternion algorithm is defined as follows:

The quaternion is defined in equation 1:
q=q₀+q₁·i+q₂·j+q₃·k (3)
with the conditions of equation 4 to equation 8.
q_{0.3}∈ (4)
i²=j²=k²=−1 (5)
i·j=−j·i=k (6)
j·k=−k·j=i (7)
k·i=−i·k=j (8)

By integrating the complex parts into a vector v and with q₀=w, equation 9 is obtained
q∈(w·v)^{T} (9)
with the conditions of equation 10 and equation 11.
w∈ [symbol] (10)
v∈ [symbol] ³ (11)

The definitions of equations 12 to 17 apply to the use of quaternions.
Conjugated quaternions

$${\underline{q}}^{k} = ( \begin{matrix}w \\ - \underline{v}\end{matrix} ) \qquad ( 12 )$$

Norm
|q|=\sqrt{w²+v·v}
Inversion

$${\underline{q}}^{- 1} = \frac{{\underline{q}}^{k}}{\vert \underline{q} \vert} \qquad ( 14 )$$

Multiplication

$${\underline{q}}_{1} \cdot {\underline{q}}_{2} = ( \begin{matrix}{w}_{1} \\ {\underline{v}}_{1}\end{matrix} ) \cdot ( \begin{matrix}{w}_{2} \\ {\underline{v}}_{2}\end{matrix} ) = ( \begin{matrix}{w}_{1} \cdot {w}_{2} + {\underline{v}}_{1} \cdot {\underline{v}}_{2} \\ {w}_{1} \cdot {\underline{v}}_{2} + {w}_{2} \cdot {\underline{v}}_{1} + {\underline{v}}_{1} \times {\underline{v}}_{2}\end{matrix} ) \qquad ( 15 )$$

Representation of a vector

$${\underline{q}}_{v} = ( \begin{matrix}0 \\ \underline{v}\end{matrix} ) \qquad ( 16 )$$

Representation of a scalar

$${\underline{q}}_{w} = ( \begin{matrix}w \\ \underline{0}\end{matrix} ) \qquad ( 17 )$$

Multiplication is especially important for the inertial object tracing. It represents rotation of a quaternion. For this purpose, a rotation quaternion is included in eq. 18.

$${\underline{q}}_{rot} = ( \begin{matrix}cos ( \frac{\vert \underline{ϕ} \vert}{2} ) \\ sin ( \frac{\vert \underline{ϕ} \vert}{2} ) \cdot \frac{\underline{ϕ}}{\vert \underline{ϕ} \vert}\end{matrix} ) \qquad ( 18 )$$

Vector φ consists of the individual rotations around the coordinate axes.

The rotation of a point or vector can now be calculated in the following way: First, the coordinates of the point or vector have to be transformed into a quaternion by means of equation 16, after which multiplication by the rotation quaternion (eq. 18) is performed. The resulting quaternion contains the rotating vector in the same notation. If the norm of a quaternion equals one, the inverted quaternion may be replaced with a conjugated quaternion (eq. 19).
q_{v′}=qᵣₒₜ·qᵥ·qᵣₒₜ [symbol] ⁻¹=qᵣₒₜ·qᵥ·qᵣₒₜ [symbol] ^{-k} (19)
due to eq. 20.
|qᵣₒₜ|=1 (20)

How can this operation be explained?φ is the normal vector to the plane, where a rotation around the angle ½φ is executed. The angle matches the value of vector φ. See FIG. 1.

FIG. 1 shows that a rotation may be performed in any plane and specification of only one angle. This also shows the particular advantages of this method. Other advantages are the reduced number of necessary parameters and trigonometric functions, which can be totally replaced by approximations for small angles. Differential equation 21 applies to the rotation with vector ω:

$$\frac{ⅆ}{ⅆ t} {\underline{q}}_{rot} = \frac{1}{2} {\underline{q}}_{rot} \cdot ( \begin{matrix}0 \\ \underline{ω}\end{matrix} ) \qquad ( 21 )$$

The concrete transformation of the quaternion algorithm is represented in FIG. 2 and is carried out in the following way: The entire calculation is carried out with the aid of unit vectors. The initial unit vectors Eₓ, E_{y} and E_{z} are determined on the basis of the initial orientation.

With the aid of equation 22 the rotation matrix is calculated from the unit vectors.

$$R = [ \begin{matrix}{( {q}_{0} )}^{2} + {( {q}_{1} )}^{2} - {( {q}_{2} )}^{2} - {( {q}_{3} )}^{2} & 2 \cdot ( {q}_{1} \cdot {q}_{2} - {q}_{0} \cdot {q}_{3} ) & 2 \cdot ( {q}_{1} \cdot {q}_{3} - {q}_{0} \cdot {q}_{2} ) \\ 2 \cdot ( {q}_{1} \cdot {q}_{2} - {q}_{0} \cdot {q}_{3} ) & {( {q}_{0} )}^{2} - {( {q}_{1} )}^{2} + {( {q}_{2} )}^{2} - {( {q}_{3} )}^{2} & 2 \cdot ( {q}_{2} \cdot {q}_{3} - {q}_{0} \cdot {q}_{1} ) \\ 2 \cdot ( {q}_{1} \cdot {q}_{3} - {q}_{0} \cdot {q}_{2} ) & 2 \cdot ( {q}_{2} \cdot {q}_{3} - {q}_{0} \cdot {q}_{1} ) & {( {q}_{0} )}^{2} - {( {q}_{1} )}^{2} - {( {q}_{2} )}^{2} + {( {q}_{3} )}^{2}\end{matrix} ] \qquad ( 22 )$$

The rotation matrix R, which is a 3×3 matrix, is calculated according to equation 22 on the basis of an initial orientation of the coordinates system related to the object, especially on the basis of so called starting unit vectors. A rotation quaternion qᵣₒₜ(k) is obtained by inverting this equation 22. With the aid of the zero quaternion, which results from the zero unit vectors, the initial quaternion is calculated via multiplication by the rotation quaternion. On the next sensing step, i.e. the next acquisition of the measured data and integration at the current infinitesimal rotation angles A, B, C, a rotation quaternion qᵣₒₜ(k) is then calculated, which will be used at this step. The quaternion qₐₖₜ(k−1) resulting from the preceding step is then multiplied by this rotation quaternion qᵣₒₜ(k) according to equation 13 in order to obtain the current quaternion of the preceding k-step, i.e. qₐₖₜ(k). The current orientation of the object can then be determined by means of this current quaternion for the just performed sensing step.

As already mentioned above, a Kalman filter algorithm can be applied in order to increase the precision of the determination or calculation of position data. The concept of Kalman filtering, in particular indirect Kalman filtering, is based on the existence of supporting information. The difference between the information obtained from the values measured by the sensors and this supporting information serves as an input signal for the Kalman filter. However, as the method and device according to the present disclosure do not obtain continuous information from a reference system, the supporting information for the determination of the position is not available in any case. Nevertheless, to enable application of indirect Kalman filtering, the use of a second parallel acceleration sensor is proposed. The difference between the sensor signals of the parallel acceleration sensors will then serve as an input signal for the Kalman filter. FIGS. 3, 4 and 5 schematically show the concept according to the present disclosure of a redundant parallel system for Kalman filtering, two sensors being arranged such that their sensitive sensor axes extend parallel to one another (FIG. 4).

Both integrations steps are advantageously included in the modeling. Thus, an error of estimation for the positioning error inevitably resulting from double integration is obtained. This is explained schematically in FIG. 5 in a feed-forward configuration as the concrete implementation of a general indirect Kalman filter.

In this concept, a first order Gauss-Markov process causes the acceleration error aided by white noise. The model is based on the fact that the positioning error is determined from the acceleration error by double integration. The outcome is equations 23 and 25.
\dot{e}ₛ(t)=eᵥ(t) 23)
\dot{e}ᵥ(t)=eₐ(t) (24)
\dot{e}ₐ(t)=β·eₐ(t)·wₐ(t) (25)

Following the general stochastic state space description of a continuous-time system model with state vector x(t) , state transition matrix φ(T), stochastic scattering matrix G and measuring noise w(t), the system equations 26 and 27 result.

$$\begin{matrix}\dot{\underline{x}} ( t ) = {ϕ}_{\underline{\_}} ( T ) \cdot \underline{x} ( t ) + {G}_{\underline{\_}} \cdot \underline{w} ( t ) & ( 26 ) \\ [ \begin{matrix}{\dot{e}}_{s} ( t ) \\ {\dot{e}}_{v} ( t ) \\ {\dot{e}}_{a} ( t )\end{matrix} ] = [ \begin{matrix}0 & 1 & 0 \\ 0 & 0 & 1 \\ 0 & 0 & - β\end{matrix} ] [ \begin{matrix}{e}_{s} ( t ) \\ {e}_{v} ( t ) \\ {e}_{a} ( t )\end{matrix} ] + [ \begin{matrix}0 & 0 & 0 \\ 0 & 0 & 0 \\ 0 & 0 & 1\end{matrix} ] [ \begin{matrix}{w}_{s} ( t ) \\ {w}_{v} ( t ) \\ {w}_{a} ( t )\end{matrix} ] & ( 27 )\end{matrix}$$

Equations 28 and 29 apply to the measuring noise w(t).
E{w(t)}=0 (28)
E{w(t)·w(t)^{T}}=Q_{d}·δ(t−t^{T}) (29)

The general stochastic state space description for the equivalent time-discrete system model results according to equations 30 and 31.

$$\begin{matrix}\underline{x} ( k + 1 \vert k ) = {ϕ}_{\underline{\_}} ( T ) \cdot \underline{x} ( k \vert k ) + {\underline{w}}_{d} ( k \vert k ) & ( 30 ) \\ [ \begin{matrix}{e}_{s} ( k + 1 \vert k ) \\ {e}_{v} ( k + 1 \vert k ) \\ {e}_{a} ( k + 1 \vert k )\end{matrix} ] = {ϕ}_{\underline{\_}} ( T ) \cdot [ \begin{matrix}{e}_{s} ( k \vert k ) \\ {e}_{v} ( k \vert k ) \\ {e}_{a} ( k \vert k )\end{matrix} ] + {\underline{w}}_{d} ( k \vert k ) & ( 31 )\end{matrix}$$

Equations 32 and 33 apply to the required time-discrete measuring equation.

$$\begin{matrix}y ( k ) = \underline{C} \cdot \underline{x} ( k ) + v ( k ) & ( 32 ) \\ y ( k ) = \underline{C} \cdot [ \begin{matrix}{e}_{s} ( k ) \\ {e}_{v} ( k ) \\ {e}_{a} ( k )\end{matrix} ] + v ( k ) & ( 33 )\end{matrix}$$

In equation 33, v(k) is a vector of a white noise process. The difference between the two sensor signals is applicable as an input value for the Kalman filter, so that equations 34 to 36 result for the measuring equation.

$$\begin{matrix}y ( k ) = Δ {e}_{a} ( k ) = [ {a}_{2} ( k ) + {e}_{a 2} ( k ) ] - [ {a}_{1} ( k ) + {e}_{a 1} ( k ) ] & ( 34 ) \\ y ( k ) = {e}_{a 2} ( k ) - {e}_{a 1} ( k ) & ( 35 ) \\ y ( k ) = [ \begin{matrix}0 & 0 & - 1\end{matrix} ] \cdot [ \begin{matrix}{e}_{s 1} ( k ) \\ {e}_{v 1} ( k ) \\ {e}_{a 1} ( k )\end{matrix} ] + [ \begin{matrix}0 \\ 0 \\ {e}_{a 2} ( k )\end{matrix} ] & ( 36 )\end{matrix}$$

In this model, eₐ₁(k) as well as eₐ₂(k) should be modeled as a first order Gauss-Markov process. The ansatz according to equation 37 serves for this purpose.
\dot{e}ₐ₂(t)=−β·eₐ₂(t)·wₐ₂(t) (37)

The equivalent time-discrete ansatz for this results from equation 38.
eₐ₂(k+1|k)=e^{-βT}·eₐ₂(k|k)·wₐ₂(k|k) (38)

If ea₂(k) is considered as a further state, the extended system model according to equations 39 and 40 results.

$$\begin{matrix}{\underline{x}}_{e} ( k + 1 \vert k ) = {{ϕ}_{\underline{\_}}}_{e} ( T ) \cdot {\underline{x}}_{e} ( k \vert k ) + {\underline{w}}_{de} ( k \vert k ) & ( 39 ) \\ [ \begin{matrix}{e}_{s 1} ( k + 1 \vert k ) \\ {e}_{v 1} ( k + 1 \vert k ) \\ \frac{{e}_{a 1} ( k + 1 \vert k )}{{e}_{a 2} ( k + 1 \vert k )}\end{matrix} ] = {{ϕ}_{\underline{\_}}}_{e} ( T ) \cdot [ \begin{matrix}{e}_{s 1} ( k \vert k ) \\ {e}_{v 1} ( k \vert k ) \\ \frac{{e}_{a 1} ( k \vert k )}{{e}_{a 2} ( k \vert k )}\end{matrix} ] + {\underline{w}}_{de} ( k \vert k ) & ( 40 )\end{matrix}$$

Here, equations 41 to 43 apply.

$$\begin{matrix}{{ϕ}_{\underline{\_}}}_{e} ( T ) = [ \begin{matrix}{ϕ}_{\underline{\_}} ( T ) & 0 \\ 0 & {ⅇ}^{- {β}_{2} \cdot T}\end{matrix} ] & ( 41 ) \\ {\underline{w}}_{de} = [ \begin{matrix} {\underline{w}}_{a 1} ( k ) \\ \underline{{\underline{w}}_{a 2} ( k )}\end{matrix} ] & ( 42 ) \\ {{Q}_{\underline{\_}}}_{de} = [ \begin{matrix}{{Q}_{\underline{\_}}}_{de} & 0 \\ 0 & {q}_{a 2}\end{matrix} ] & ( 43 )\end{matrix}$$

Equations 44 to 47 apply to the extended measurement model.

$$\begin{matrix}y ( k ) = [ {a}_{2} ( k ) + {e}_{a 2} ( k ) ] - [ {a}_{1} ( k ) + {e}_{a 1} ( k ) ] & ( 44 ) \\ y ( k ) = {e}_{a 2} ( k ) - {e}_{a 1} ( k ) & ( 45 ) \\ y ( k ) = \underline{C} \cdot \underline{x} ( k ) + v ( k ) & ( 46 ) \\ y ( k ) = [ \begin{matrix}0 & 0 & - 1 \vert & 1\end{matrix} ] \cdot [ \begin{matrix}{e}_{s 1} ( k ) \\ {e}_{v 1} ( k ) \\ \frac{{e}_{a 1} ( k )}{{e}_{a 2} ( k )}\end{matrix} ] + v ( k ) & ( 47 )\end{matrix}$$

This measurement equation is a perfect and hence flawless measurement, i.e. there is no measurement noise v(k). Thus, modeling according to equation 48 is required.
R(k)=r(k)=0 (48)

Hence, the covariance matrix R of the measuring noise is singular, i.e. R⁻¹ is non-existent. The existence of R⁻¹ is a sufficient but not necessary condition for the stability and/or stochastic observability of the Kalman filter. There are now two possibilities of reacting to singularity:

1. Using R=0. The filter may be stable. As only short-term stability is required in this case, long-term stability can be dispensed with.

2. Using a reduced scanner.

Variance R=0 is used in this concept. The filters used are sufficiently stable with this method.

The Kalman filter equations for the one-dimensional discrete system result from equations 49 to 50.

Determination of the Kalman enhancement according to equation 49.
K(k+1|)=P(k+1|k)·C^{T}(k) [symbol] C(k)·P(k+1|k)·C^{T}(k)+R(k) [symbol] ⁻¹ (49)

Update of the state prediction according to equation 50.
\hat{x}(k+1|k+1)=\hat{x}(k+1|k)+K(k+1)·\tilde{y}(k+1) (50)

Where equation 51 applies.
\tilde{y}(k)=y(k)−ŷ(k)=y(k)−C(k)·\hat{x}(k) (51)

Update of the covariance matrix of the error of estimation according to equations 52 and/or 53.

$$\begin{matrix}\begin{matrix}{P}_{\underline{\_}} ( k + 1 \vert k + 1 ) = ( {I}_{\underline{\_}} + {K}_{\underline{\_}} ( k + 1 ) \cdot {\underline{C}}^{T} ( k ) ) \cdot {P}_{\underline{\_}} ( k + 1 \vert k ) \cdot \\ {( {I}_{\underline{\_}} - {K}_{\underline{\_}} ( k + 1 ) \cdot {\underline{C}}^{T} ( k ) )}^{T} + {K}_{\underline{\_}} ( k + 1 ) \cdot R ( k ) \cdot {{K}_{\underline{\_}}}^{{T}^{′}} ( k + 1 )\end{matrix} & ( 52 ) \\ {P}_{\underline{\_}} ( k + 1 \vert k + 1 ) = ( {I}_{\underline{\_}} - {K}_{\underline{\_}} ( k + 1 ) \cdot {\underline{C}}^{T} ( k ) ) \cdot {P}_{\underline{\_}} ( k + 1 \vert k ) & ( 53 )\end{matrix}$$

Determination of the predictive value of the system state according to equation 54.
\hat{x}(k+2|k+1)=φ(T)·\hat{x}(k+1|k+1) (54)

Determination of the predictive value of the covariance matrix of the error of estimation according to equation 55.
P(k+2|k+1)=φ(T)·P(k+1|k+1)·φ^{T}(T)+Q(k) 55)

Thus, the filter cycle is complete and restarts for the next measurement. The filter operates recursively, the predictive steps and corrections being filtered again on each measurement.

The applied system describes a three-dimensional translation in three orthogonal space axes. These translations are described by path s, speed v and acceleration a. An additional acceleration sensor for each space direction likewise provides acceleration information for indirect Kalman filtering.

The basic algorithm of the design is displayed in FIG. 5. The actual measuring signal for each space axis is provided by an acceleration sensor as acceleration a. Aided by the supporting information as a sensor signal from the second acceleration sensor for each space axis, the Kalman filter algorithm provides an estimated value for the deviation of the acceleration signal ea for the three space directions x, y and z.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

Further characteristics, particularities and advantages of the present disclosure result from the patent claims and drawings that will be described hereinafter. The drawings show:

FIG. 1 is a diagram of the rotation of a vector by means of quaternions;

FIG. 2 is a flow diagram that illustrates the application of the quaternion algorithm;

FIG. 3 is a flow diagram that illustrates the execution of the method according to the present disclosure;

FIG. 4 is a schematic illustration of an acceleration sensor and a redundant acceleration sensor arranged parallel to it;

FIG. 5 is a schematic indication of a Kalman filter with INS error modeling in a feed-forward configuration; and

FIG. 6 is a schematic illustration of the results of the application of Kalman filtering.

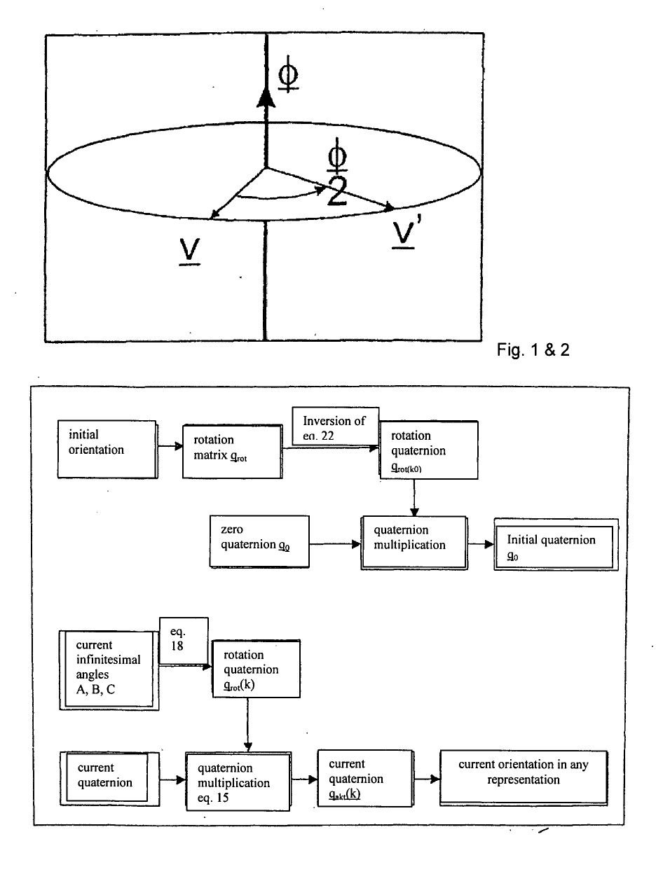

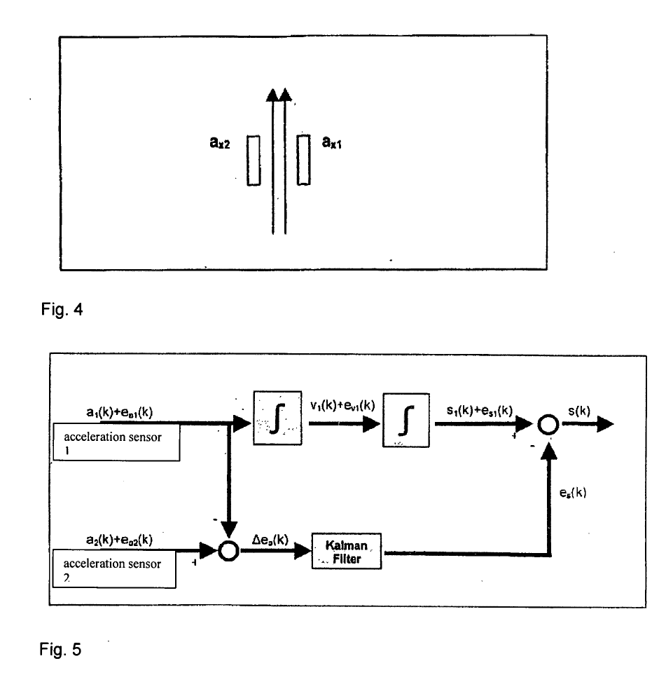

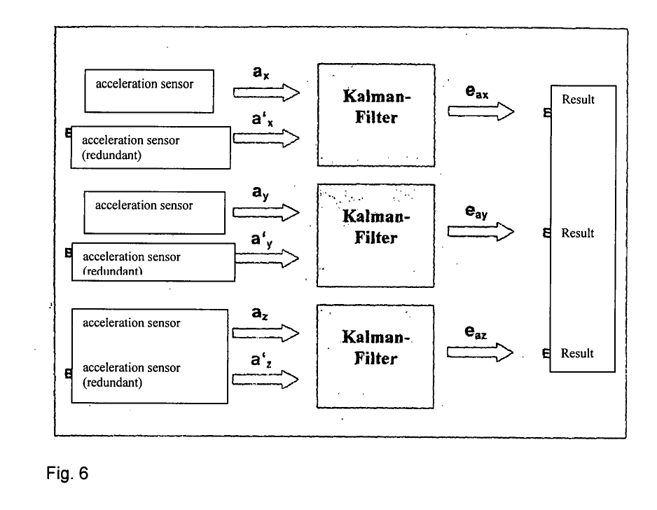

FIGS. 1, 2, and 4 to 6 have already been explained above.

### DETAILED DESCRIPTION

The following description is merely exemplary in nature and is not intended to limit the present disclosure, application, or uses.

As already mentioned, at the beginning of object tracing, a sensor device is attached to the object to be positioned in the predetermined place. The object is then brought to a standstill in the room and referenced with a fixed coordinates system, e.g. the operating table, such that the angle and accelerations determined via the signals from the rotational speed sensors and acceleration sensors are set to 0. During an absolute rest period of the object, an offset value is determined, which is contemplated at each sensing step, i.e. at each data acquisition. This is a drift vector, whose components comprise the determined sensor offset values.

Especially advantageous is that every time a rest period of the sensor device is detected and applied, the sensor offset values are again determined and applied to the next calculation of the position and orientation.

The aforesaid compensation matrix is further determined, which corresponds to or should exactly compensate for an axial deviation of the rotational speed sensors and an assumed orientation to each other and to a housing of the sensor device.

The above embodiments are applicable to the second sensor device, which is to be attached to the patient.

On positioning and orientation, the sensor signals are acquired and converted within consecutive time intervals by simple or double integration into infinitesimal rotation angles and position data at a sensing rate of 10 to 30 Hz, especially 20 Hz. In this process, the compensation matrix for the non-orthogonality of the rotational speed sensors is contemplated in order to achieve increased precision in the determination of the orientation.

On the basis of the infinitesimally small angular variations determined from the measuring signals of the three rotational speed sensors, the orientation of the twisted coordinate system of the object with respect to the reference coordinate system can now be determined by indicating three angles in application of Euler's method. Instead, it proves to be advantageous if a quaternion algorithm of the aforementioned type is used to determine the orientation. Thus, instead of three consecutive rotations, a single transformation can be assumed, which may further improve the precision of the orientation of the object system obtained in this way.

The orientation of the object in the room is given by the result of the quaternion algorithm execution.

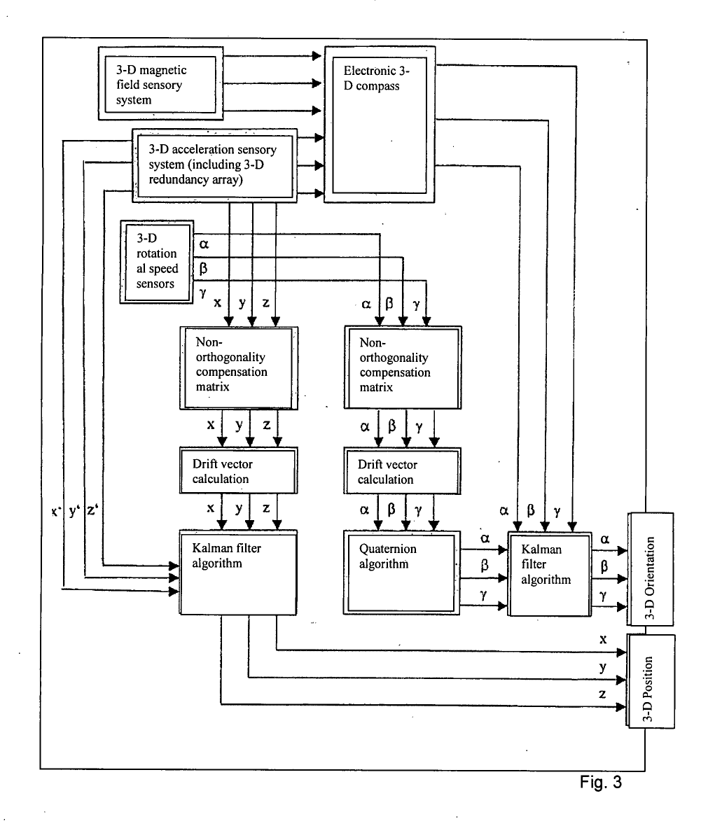

As indicated in FIG. 3, however, it is possible to determine the magnetic field acting at any desired time on the object by means of further sensors, e.g. a three-dimensional magnetic field sensor system. Additionally, it is possible to provide three-dimensional acceleration sensors to measure gravitational acceleration. The measuring signals of the magnetic field and acceleration sensors can be combined into an electronic three-dimensional compass, which can indicate the orientation of the object in the room with great precision if parasitic effects are absent, preferably if the measured values are taken during a rest period of the object. The obtained space orientation of the object can be used as supporting information for the orientation that was obtained only via the signals of the three rotational speed sensors. In the first instance, the measurement signals of the magnetic field and acceleration sensors are examined for interferences. If this is not the case, they are taken as the supporting information to be contemplated during performance of the method as compared with the orientation information obtained from the three rotational speed sensors. A Kalman filter algorithm is used advantageously to this end. This is an estimation algorithm, in which information on the orientation of the object determined by the aforementioned three-dimensional compass is used as correct supporting information when it is compared with the information on the orientation obtained by the rotational speed sensors.

As described above in detail, the measured values of the acceleration sensors can also be improved by the application of Kalman filtering by preferably providing a redundant acceleration sensor for each acceleration sensor, arranged parallel to them, as a replacement for supporting information that is accessible from elsewhere. With the aid of this additional information in the form of the measured value signal from the second acceleration sensor for each space axis, an estimated value for this faulty deviation from the measured acceleration value signal for the related space orientation can be determined.

It should be noted that the disclosure is not limited to the embodiment described and illustrated as examples. A large variety of modifications have been described and more are part of the knowledge of the person skilled in the art. These and further modifications as well as any replacement by technical equivalents may be added to the description and figures, without leaving the scope of the protection of the disclosure and of the present patent.

What is claimed is:

1. A method for navigating and positioning an object relative to a patient during surgery in an operating room, characterized in that the position and orientation in the room of both the object and the patient or of a relevant area of the patient relative to a referencing framework is determined quasi continuously according to a sensing rate by means of three-dimensional inertial sensors, and that from this the current position and orientation of the object relative to the patient is determined, that this position and orientation are compared with a desired, predetermined position and orientation, and that there is an indication as to how the position of the object should be modified in order to be placed in the desired predetermined position and orientation.

2. The method according to claim 1, characterized in that the sensing rate is about 10-50 Hz.

3. The method according to claim 1, characterized in that the sensing rate is about 10-40 Hz.

4. The method according to claim 1, characterized in that the sensing rate is about 10-30 Hz.

5. The method according to claim 1, characterized in that the sensing rate is about 15-25 Hz.

6. An apparatus for the implementation of a method for navigating and positioning an object relative to a patient during surgery, the apparatus comprising a first sensor device with acceleration and rotational speed sensors that is attachable to and again removable from a first predetermined area of the object, and a second sensor device with acceleration and rotational speed sensors that is attachable to and again removable from a patient, a memory, where the desired predetermined position and orientation of the object relative to the patient is stored, and means of calculation to determine the position and orientation from the measured sensor values, and means of calculation to compare the determined position and orientation from the predetermined position and orientation, and indication means to indicate how the position of the object should be modified in order to be placed in the desired predetermined position and orientation.

7. The apparatus according to claim 6, characterized in that the measured values of the sensor device can be acquired and processed at a sensing rate of about 10-50 Hz.

8. The apparatus according to claim 6, characterized in that the measured values of the sensor device can be acquired and processed at a sensing rate of about 10-40 Hz.

9. The apparatus according to claim 6, characterized in that the measured values of the sensor device can be acquired and processed at a sensing rate of about 10-30 Hz.

10. The apparatus according to claim 6, characterized in that the measured values of the sensor device can be acquired and processed at a sensing rate of about 15-25 Hz.

11. The apparatus according to claim 6, characterized in that the sensor devices have an orientation aid, which allows fastening to at least one of the object and the patient.

12. The apparatus according to claim 6, characterized in that the sensor devices have means of fastening for the removable attachment of the sensor device to the object and the patient.

13. The apparatus according to claim 6, characterized in that the first and the second sensor device comprise three acceleration sensors, whose signals may be used for the calculation of translational movements, and also three rotational speed sensors, whose measured values may be used for the determination of the orientation in the room.

14. The apparatus according to claim 6, characterized in that the means of calculation include execution of a quaternion algorithm.

15. The apparatus according to claim 1, characterized in that the means of calculation include application of a compensation matrix that is determined and stored prior to the start of positioning, said compensation matrix allowing for a deviation of the axial orientation of the three rotational speed sensors from an assumed orientation of the axes toward each other and compensating for errors resulting from the calculation of the rotation angles.

16. The apparatus according to claim 6, characterized in that the means of calculation include executing a Kalman filter algorithm.

17. The apparatus according to claim 6 further comprising magnetic field sensors for the determination of the space orientation of the object.

18. The apparatus according to claim 17, characterized in that means for comparing the space orientation determined from the values measured by the magnetic field sensors with the space orientation determined by the values measured by the rotational speed sensors are provided.

19. The apparatus according to claim 17, characterized in that means for comparing the space orientation determined from the values measured by a magnetic field sensor with the space orientation determined by the values measured by a gravitational acceleration sensor are provided.

20. The apparatus according to claim 6, characterized in that for each acceleration sensor a redundant acceleration sensor arranged parallel to it is provided for the implementation of Kalman filtering.
